(19) United States
(12) Patent Application Publication
(10) Pub. No.: US 2022/0000182 A1
(43) Pub. Date: Jan. 6, 2022

(54) VAPORIZER DEVICES AND ACCESSORIES WITH INTEGRATED SENSORS

(71) Applicant: JUUL Labs, Inc., San Francisco, CA (US)

(72) Inventors: Ariel Atkins, San Francisco, CA (US); Nikhil Bhat, Fremont, CA (US); Steven Christensen, Burlingame, CA (US); Joseph R. Fisher, JR., Santa Cruz, CA (US); Nicholas J. Hatton, Oakland, CA (US); Vittal Kadapakkam, San Francisco, CA (US); Andrew L. Murphy, San Francisco, CA (US); James W. Pace, San Francisco, CA (US); Bryan White, Pleasanton, CA (US)

(21) Appl. No.: 17/480,050

(22) Filed: Sep. 20, 2021

Related U.S. Application Data
(63) Continuation of application No. PCT/US20/25854, filed on Mar. 31, 2020.
(60) Provisional application No. 62/826,669, filed on Mar. 29, 2019.

(51) Int. Cl. A24F 40/51; H04W 4/38; A24F 40/60; A24F 40/65; A24F 40/53; G16H 40/67

(52) U.S. Cl. CPC A24F 40/51; H04W 4/38; A24F 40/60; A61B 5/4845; A24F 40/53; G16H 40/67; A24F 40/65

(57) ABSTRACT

A vaporizer system may include a vaporizer device and a vaporizer accessory configured to couple to the vaporizer device. The vaporizer accessory may include a sensor configured to continuously and passively monitor a biomarker of the user. The biomarker includes a concentration of carbon monoxide in the user's blood. Related systems and methods are also described.

### CROSS-REFERENCE TO RELATED APPLICATIONS

This current application claims priority to U.S. Provisional Patent Application No. 62/826,669, filed on Mar. 29, 2019 and entitled “Vaporizer Device or accessory with Integrated Sensors,” the disclosure of which is incorporated herein by reference in its entirety, to the extent permissible.

### FIELD

The subject matter described herein relates to vaporizer devices, and more particularly, to vaporizer devices and vaporizer device accessories having integrated sensors.

### BACKGROUND

Vaporizer devices, which can also be referred to as vaporizers, electronic vaporizer devices, or e-vaporizer devices, can be used for delivery of an aerosol (for example, a vapor-phase and/or condensed-phase material suspended in a stationary or moving mass of air or some other gas carrier) containing one or more active ingredients by inhalation of the aerosol by a user of the vaporizing device. For example, electronic nicotine delivery systems (ENDS) include a class of vaporizer devices that are battery powered and that can be used to simulate the experience of smoking, but without burning of tobacco or other substances. Vaporizers are gaining increasing popularity both for prescriptive medical use, in delivering medicaments, and for consumption of tobacco, nicotine, and other plant-based materials. Vaporizer devices can be portable, self-contained, and/or convenient for use.

In use of a vaporizer device, the user inhales an aerosol, colloquially referred to as “vapor,” which can be generated by a heating element that vaporizes (e.g., causing a liquid or solid to at least partially transition to the gas phase) a vaporizable material, which can be liquid, a solution, a solid, a paste, a wax, and/or any other form compatible for use with a specific vaporizer device. The vaporizable material used with a vaporizer can be provided within a cartridge (e.g., a separable part of the vaporizer device that contains vaporizable material) that includes an outlet (e.g., a mouthpiece) for inhalation of the aerosol by a user.

To receive the inhalable aerosol generated by a vaporizer device, a user may, in certain examples, activate the vaporizer device by taking a puff, by pressing a button, and/or by some other approach. A puff as used herein can refer to inhalation by the user in a manner that causes a volume of air to be drawn into the vaporizer device such that the inhalable aerosol is generated by a combination of the vaporized vaporizable material with the volume of air.

An approach by which a vaporizer device generates an inhalable aerosol from a vaporizable material involves heating the vaporizable material in a vaporization chamber (e.g., a heater chamber) to cause the vaporizable material to be converted to the gas (or vapor) phase. A vaporization chamber can refer to an area or volume in the vaporizer device within which a heat source (e.g., a conductive, convective, and/or radiative heat source) causes heating of a vaporizable material to produce a mixture of air and vaporized vaporizable material to form a vapor for inhalation of the vaporizable material by a user of the vaporization device.

Some vaporizer users, particularly those participating in a cessation program for smoking combustible cigarettes (e.g., smokers who want to quit smoking), may wish to monitor their CO (carbon monoxide) levels, among other biomarkers. However, breathalyzer devices, pulse oximeters, blood sampling, other devices, and methods of actively measuring and monitoring the users' CO levels require users to take active steps outside of the ordinary use of the vaporizer device. Active methods of measuring and monitoring users' CO levels may discourage the users from tracking their CO levels.

### SUMMARY

Biological metrics (e.g., vital signs and/or other biomarkers) can be collected by way of sensors connected to, or communicating with, a vaporizer device or vaporizer device accessory for the purpose of detecting whether a person is improving their smoking habits or lifestyle. The collected data can be provided in a meaningful way to the user and/or a person monitoring the user. The information can be based on determining CO levels during a user's inhalation and/or exhalation to determine, for example, whether the person is smoking cigarettes, how many cigarettes the person is smoking, and/or the like. The sensors can also monitor and/or detect heartrate, diet, blood pressure, blood-level nicotine, sleeping habits, and/or the like. A report or alert can be generated based on one or more, or an analysis of a combination, of any of the factors measured by or through the sensors.

In certain aspects of the current subject matter, challenges associated with the limitations of conventional vaporizer devices and vaporizer systems can be addressed by the inclusion of one or more of the features described herein or comparable/equivalent approaches as would be understood by one of ordinary skill in the art. Aspects of the current subject matter are related to a vaporizer accessories including one or more sensors (e.g., an optical sensor). In some implementations, a vaporizer system can include a vaporizer device and a vaporizer accessory configured to couple to the vaporizer device. In some implementations, the vaporizer accessory includes a sensor that continuously (e.g., periodically, according to a specific schedule and/or based on additional triggers) and passively monitors one or more biomarkers of the user. The one or more biomarkers can include a concentration of carbon monoxide in the user's blood.

In one aspect, a wireless communication device includes a wireless communication interface and a controller configured to perform multiple operations. Those operation include receiving sensor data from a vaporizer device or accessory, determining whether the user smoked a combustible cigarette based on the sensor data, and providing an indication that use of combustible cigarettes has been detected in response to determining that the user smoked a combustible cigarette. The sensor data are derived from one or more sensors of the vaporizer device or accessory and/or are based on biomarkers of a user of the vaporizer device or accessory.

In another interrelated aspect, a vaporizer device system includes a controller configured to, among other possible operations, receive sensor data from a vaporizer device or an accessory (where, as above, the sensor data are derived from one or more sensors of the vaporizer device or accessory and/or are based on biomarkers of a user of the vaporizer device or accessory, determine whether the user smoked a combustible cigarette based on the sensor data, and provide an indication that use of combustible cigarettes has been detected in response to determining that the user smoked a combustible cigarette.

In optional variations, the vaporizer device includes a wireless communication device and the controller. In other variations, the vaporizer device system includes the vaporizer device and a wireless communication device separate from the vaporizer device, where the wireless communication device includes the controller.

In some variations, one or more of the following features can optionally be included in any feasible combination. In some aspects, the vaporizer accessory can be physically coupled to the vaporizer device. In some aspects, the concentration of carbon monoxide is measured from a breath of the user when the user uses the vaporizer device. In some aspects, the concentration of carbon monoxide is measured from the user's blood when the sensor contacts skin of the user when the user uses the vaporizer device.

In some variations, the vaporizer accessory is configured to wirelessly communicate with the vaporizer device. In some aspects, the vaporizer accessory is configured to be worn by the user. In some aspects, the vaporizer system further includes a user device. The vaporizer accessory may transmit the monitored biomarker to the user device to thereby send an indicator to the user.

In some variations, a vaporizer device includes an outer surface and a sensor positioned along the outer surface. The sensor may contact skin of a user when the user uses the vaporizer device. The sensor may continuously measure a biomarker of the user when the user uses the vaporizer device. In some aspects, the biomarker includes a concentration of carbon monoxide in the user's blood.

In some variations, a method of guiding a user participating in a nicotine cessation program includes monitoring, passively and continuously by a sensor of a vaporizer accessory, a biomarker of a user. The biomarker may include a concentration of carbon monoxide of the user. The method may also include transmitting, by the vaporizer accessory, the monitored biomarker to a user device in wireless communication with the vaporizer accessory. The user device may include a user interface. The method may further include providing an indicator to the user via the user interface based on the monitored biomarker.

In optional variations, one or more of the following features may be present in any feasible combination. The one or more biomarkers may include a heartbeat, a heart rate, a perspiration, a pupil dilation, a body temperature, a blood sugar level, a blinking frequency, a blood carbon monoxide level, a breath carbon monoxide level, a blood pressure, a blood oxygen level, a breathing rate, a location, a blood alcohol level, and/or a motion.

The controller may be further configured to determine, based at least on the sensor data, whether the one or more biomarkers of the user exceed a threshold value, and in response to determining that the one or more biomarkers of the user exceed the threshold value, provide, to the user, a second indication that the one or more biomarkers exceed the threshold value.

The controller may be further configured to receive, from the user, one or more inputs indicative of the use of combustible cigarettes, and detect, based at least on the one or more inputs, the use of combustible cigarettes by the user.

The one or more inputs may include a second indication that the user used combustible cigarettes, a quantity of combustible cigarettes used, and/or a time when combustible cigarettes are used.

The second indication may be received via a user ser interface displayed at the accessory associated with the vaporizer device.

The first indication may be provided via a user interface displayed at the accessory associated with the vaporizer device.

The use of combustible cigarettes may be detected based at least on the one or more biomarkers exceeding a threshold value.

The threshold value may include a blood carbon monoxide level of 3 percent and/or 9 parts per million.

The one or more sensors may include an optical sensor, a touch activated sensor, an ambient air sensor, an inhalation sensor, an exhalation sensor, a gas sensor, a photoionization detector, an infrared sensor, an ultrasonic sensor, an electrochemical gas sensor, and/or a semiconductor sensor.

The details of one or more variations of the subject matter described herein are set forth in the accompanying drawings and the description below. Other features and advantages of the subject matter described herein will be apparent from the description and drawings, and from the claims. The claims that follow this disclosure are intended to define the scope of the protected subject matter.

### BRIEF DESCRIPTION OF THE DRAWINGS

The accompanying drawings, which are incorporated into and constitute a part of this specification, show certain aspects of the subject matter disclosed herein and, together with the description, help explain some of the principles associated with the disclosed implementations. In the drawings:

FIG. 1A illustrates a block diagram of a vaporizer consistent with implementations of the current subject matter;

FIG. 1B illustrates a top view of an implementation of the vaporizer of FIG. 1A showing a cartridge separated from a vaporizer device body;

FIG. 2 illustrates communication between a vaporizer, a user device, and a server consistent with implementations of the current subject matter;

FIG. 3A illustrates an exploded view of an example vaporizer device with a vaporizer accessory having an integrated sensor, consistent with implementations of the current subject matter;

FIG. 3B illustrates an example vaporizer device with an integrated sensor, consistent with implementations of the current subject matter;

FIG. 3C illustrates an example vaporizer accessory having an integrated sensor, consistent with implementations of the current subject matter;

FIG. 4 illustrates a functional block diagram of a user device for implementing features consistent with the described subject matter, consistent with some example implementations; and

FIG. 5 illustrates an example method of guiding a user participating in a nicotine cessation program, consistent with implementations of the current subject matter.

When practical, similar reference numbers denote similar structures, features, or elements.

### DETAILED DESCRIPTION

Implementations of the current subject matter include methods, apparatuses, articles of manufacture, and systems relating to vaporization of one or more materials for inhalation by a user. Example implementations include vaporizer devices and systems including vaporizer devices. The term “vaporizer device” as used in the following description and claims refers to any of a self-contained apparatus, an apparatus that includes two or more separable parts (for example, a vaporizer body that includes a battery and other hardware, and a cartridge that includes a vaporizable material), and/or the like. A “vaporizer system,” as used herein, can include one or more components, such as a vaporizer device. Examples of vaporizer devices consistent with implementations of the current subject matter include electronic vaporizers, electronic nicotine delivery systems (ENDS), and/or the like. Such vaporizers are generally portable, hand-held devices that heat (such as by convection, conduction, radiation, and/or some combination thereof) a vaporizable material to provide an inhalable dose of the material. The vaporizable material used with a vaporizer device can be provided within a cartridge (e.g., a separable part of the vaporizer that contains the vaporizable material in a reservoir or other container and that can be refillable when empty, or disposable in favor of a new cartridge containing additional vaporizable material of a same or different type). A vaporizer device can be a cartridge-using vaporizer device, a cartridge-less vaporizer device, or a multi-use vaporizer device capable of use with or without a cartridge. For example, a vaporizer device can include a heating chamber (e.g., an oven or other region in which material is heated by a heating element) configured to receive a vaporizable material directly in the heating chamber and also to receive a cartridge or other replaceable device having a reservoir, a volume, or the like for at least partially containing a usable amount of vaporizable material.

In various implementations, a vaporizer device can be configured for use with a liquid vaporizable material (e.g., a carrier solution in which an active and/or inactive ingredient(s) are suspended or held in solution, or a liquid form of the vaporizable material itself), a paste, a wax, and/or a solid vaporizable material. A solid vaporizable material can include a plant material that emits some part of the plant material as the vaporizable material (e.g., such that some part of the plant material remains as waste after the vaporizable material is vaporized for inhalation by a user) or optionally can be a solid form of the vaporizable material itself (e.g., a “wax”) such that all of the solid material can eventually be vaporized for inhalation. A liquid vaporizable material can likewise be capable of being completely vaporized or can include some part of the liquid material that remains after all of the material suitable for inhalation has been vaporized.

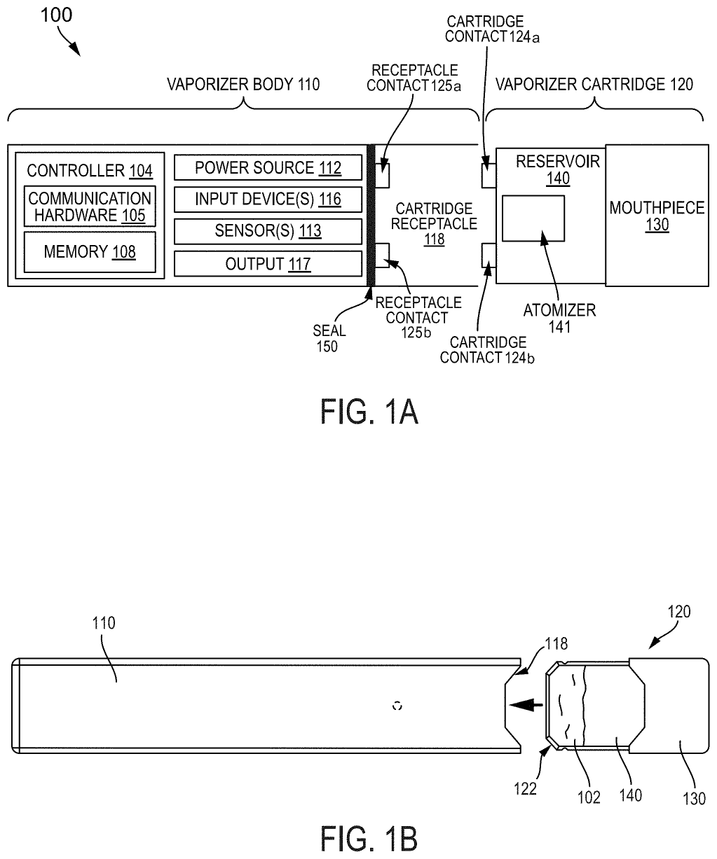

Referring to the block diagram of FIG. 1A, a vaporizer device 100 can include a power source 112 (such as a battery, which can be a rechargeable battery), and a controller 104 (e.g., a processor, circuitry, and/or the like, capable of executing logic) for controlling delivery of heat to an atomizer 141 to cause a vaporizable material 102 to be converted from a condensed form (e.g., a solid, a liquid, a solution, a suspension, a part of an at least partially unprocessed plant material, etc.) to the gas phase. The controller 104 can be part of one or more printed circuit boards (PCBs) consistent with certain implementations of the current subject matter. After conversion of the vaporizable material to the gas phase, and depending on the type of vaporizer, the physical and chemical properties of the vaporizable material, and/or other factors, at least some of the gas-phase vaporizable material can condense to form particulate matter in at least a partial local equilibrium with the gas phase as part of an aerosol, which can form some or all of an inhalable dose provided by the vaporizer device 100 for a given puff on the vaporizer device 100. It should be appreciated that the interplay between gas and condensed phases in an aerosol generated by a vaporizer device 100 can be complex and dynamic, due to factors such as ambient temperature, relative humidity, chemistry, flow conditions in airflow paths (both inside the vaporizer and in the airways of a human or other animal), mixing of the gas-phase or aerosol-phase vaporizable material with other air streams, etc. which can affect one or more physical parameters of an aerosol. In some vaporizer devices, and particularly for vaporizer devices configured for delivery of volatile vaporizable materials, the inhalable dose can exist predominantly in the gas phase (e.g., formation of condensed phase particles can be very limited).

The atomizer 141 in the vaporizer device 100 can be configured to vaporize a vaporizable material 102. The vaporizable material 102 can be a liquid. Examples of the vaporizable material 102 include neat liquids, suspensions, solutions, mixtures, and/or the like. The atomizer 141 can include a wicking element (e.g., a wick, not shown in FIG. 1A) configured to convey an amount of the vaporizable material 102 to a part of the atomizer 141 that includes a heating element (not shown in FIG. 1A).

For example, the wicking element can be configured to draw the vaporizable material 102 from a reservoir 140 configured to contain (and that may in use contain) the vaporizable material 102, such that the vaporizable material 102 can be vaporized by heat delivered from a heating element. The wicking element can also optionally allow air to enter the reservoir 140 and replace the volume of vaporizable material 102 removed. In some implementations of the current subject matter, capillary action can pull vaporizable material 102 into the wick for vaporization by the heating element, and air can return to the reservoir 140 through the wick to at least partially equalize pressure in the reservoir 140. Other approaches to allowing air back into the reservoir 140 to equalize pressure are also within the scope of the current subject matter. As used herein, the terms “wick” or “wicking element” include any material capable of causing fluid motion via capillary pressure.

The heating element can be or include one or more of a conductive heater, a radiative heater, and/or a convective heater. One type of heating element is a resistive heating element, which can be constructed of or at least include a material (e.g., a metal or alloy, for example a nickel-chromium alloy, or a non-metallic resistor) configured to dissipate electrical power in the form of heat when electrical current is passed through one or more resistive segments of the heating element. In some implementations of the current subject matter, the atomizer 141 can include a heating element that includes a resistive coil or other heating element wrapped around, positioned within, integrated into a bulk shape of, pressed into thermal contact with, or otherwise arranged to deliver heat to a wicking element, to cause the vaporizable material 102 drawn by the wicking element from a reservoir 140 to be vaporized for subsequent inhalation by a user in a gas and/or a condensed (e.g., aerosol particles or droplets) phase. Other wicking elements, heating elements, and/or atomizer assembly configurations are also possible.

Certain vaporizer devices may, additionally or alternatively, be configured to create an inhalable dose of gas-phase and/or aerosol-phase vaporizable material 102 via heating of the vaporizable material 102. The vaporizable material 102 can be a solid-phase material (such as a wax or the like) or plant material (e.g., tobacco leaves and/or parts of tobacco leaves) containing the vaporizable material. In such vaporizer devices, a resistive heating element can be part of, or otherwise incorporated into or in thermal contact with, the walls of an oven or other heating chamber into which the vaporizable material 102 is placed. Alternatively, a resistive heating element or elements can be used to heat air passing through or past the vaporizable material 102, to cause convective heating of the vaporizable material 102. In still other examples, a resistive heating element or elements can be disposed in intimate contact with plant material such that direct conductive heating of the plant material occurs from within a mass of the plant material, as opposed to only by conduction inward from walls of an oven.

The heating element can be activated in association with a user puffing (e.g., drawing, inhaling, etc.) on a mouthpiece 130 of the vaporizer device 100 to cause air to flow from an air inlet, along an airflow path that passes the atomizer 141 (e.g., wicking element and heating element). Optionally, air can flow from an the atomizer 141 through one or more condensation areas or chambers, to an air outlet in the mouthpiece 130. Incoming air moving along the airflow path moves over or through the atomizer 141, where vaporizable material 102 in the gas phase is entrained into the air. The heating element can be activated via the controller 104, which can optionally be a part of a vaporizer body 110 as discussed herein, causing current to pass from the power source 112 through a circuit including the resistive heating element, which is optionally part of a vaporizer cartridge 120 as discussed herein. As noted herein, the entrained vaporizable material 102 in the gas phase can condense as it passes through the remainder of the airflow path such that an inhalable dose of the vaporizable material 102 in an aerosol form can be delivered from the air outlet (e.g., in a mouthpiece 130) for inhalation by a user.

Activation of the heating element can be caused by automatic detection of a puff based on one or more signals generated by one or more sensors 113, such as for example a pressure sensor or sensors disposed to detect pressure along the airflow path relative to ambient pressure (or optionally to measure changes in absolute pressure), a motion sensor or sensors (for example, an accelerometer) of the vaporizer device 100, a flow sensor or sensors of the vaporizer device 100, a capacitive lip sensor of the vaporizer device 100, detection of interaction of a user with the vaporizer device 100 via one or more input devices 116 (e.g., buttons or other tactile control devices of the vaporizer device 100), receipt of signals from a computing device in communication with the vaporizer device 100, and/or via other approaches for determining that a puff is occurring or imminent.

As discussed herein, the vaporizer device 100 consistent with implementations of the current subject matter can be configured to connect (e.g., wirelessly or via a wired connection) to a computing device (or optionally to two or more computing devices) in communication with the vaporizer device 100. To this end, the controller 104 can include communication hardware 105. The controller 104 can also include a memory 108. A computing device can be a component of a vaporizer system that also includes the vaporizer device 100, and can include its own hardware for communication, which can establish a wireless communication channel with the communication hardware 105 of the vaporizer device 100. For example, a computing device used as part of a vaporizer system can include a general-purpose computing device (such as a smartphone, a tablet, a personal computer, some other portable device such as a smartwatch, or the like) that executes software to produce a user interface for enabling a user to interact with the vaporizer device 100. In example implementations of the current subject matter, such a device used as part of a vaporizer system can be a dedicated piece of hardware such as a remote control or other wireless or wired device having one or more physical or soft (i.e., configurable on a screen or other display device and selectable via user interaction with a touch-sensitive screen or some other input device like a mouse, pointer, trackball, cursor buttons, or the like) interface controls. The vaporizer device 100 can also include one or more outputs 117 or devices for providing information to the user. For example, the outputs 117 can include one or more light emitting diodes (LEDs) configured to provide feedback to a user based on a status and/or mode of operation of the vaporizer device 100.

In implementations in which a computing device provides signals related to activation of the resistive heating element, control, or other functions of a vaporizer device 100, the computing device can execute one or more computer instruction sets to provide a user interface, for underlying data handling, and/or the like. In one example, detection by the computing device of user interaction with one or more user interface elements can cause the computing device to signal the vaporizer device 100 to activate the heating element to reach an operating temperature for creation of an inhalable dose of vapor/aerosol. Functions of the vaporizer device 100 can be controlled by interaction of a user with a user interface on a computing device in communication with the vaporizer device 100.

The temperature of a resistive heating element of the vaporizer device 100 can depend on a number of factors, including an amount of electrical power delivered to the resistive heating element and/or a duty cycle at which the electrical power is delivered, conductive heat transfer to other parts of the electronic vaporizer device 100 and/or to the environment, latent heat losses due to vaporization of the vaporizable material 102 from the wicking element and/or the atomizer 141 as a whole, and convective heat losses due to airflow (e.g., air moving across the heating element or the atomizer 141 as a whole when a user puffs on the vaporizer device 100). As noted herein, to reliably activate the heating element or heat the heating element to a desired temperature, the vaporizer device 100 may, in some implementations of the current subject matter, make use of signals from the sensor(s) 113 (for example, a pressure sensor) to determine when a user is puffing on the mouthpiece 130. The sensor(s) 113 can be positioned in the airflow path and/or can be connected (for example, by a passageway or other path) to an airflow path containing an inlet for air to enter the vaporizer device 100 and an outlet via which the user inhales the resulting vapor and/or aerosol such that the sensor(s) 113 experience changes (for example, pressure changes) concurrently with air passing through the vaporizer device 100 from the air inlet to the air outlet. In some implementations of the current subject matter, the heating element can be activated in association with a user's puff, for example by automatic detection of the puff, or by the sensor(s) 113 detecting a change (such as a pressure change) in the airflow path. In some implementations, activation of the heating element may be disabled if signals from the sensor(s) 113 indicate a potential issue with operation of the vaporizer device 100.

The sensor(s) 113 can be positioned on or coupled (e.g., electrically or electronically connected, either physically or via a wireless connection) to the controller 104 (e.g., a printed circuit board assembly or other type of circuit board). To take measurements accurately and maintain durability of the vaporizer device 100, it can be beneficial to provide a seal 150 resilient enough to separate an airflow path from other parts of the vaporizer device 100. The seal 150, which can be a gasket, can be configured to at least partially surround one or more of the sensor(s) 113 such that connections of the sensor(s) 113 to the internal circuitry of the vaporizer device 100 are separated from a part of the sensor(s) 113 exposed to the airflow path. In an example of a cartridge-based vaporizer, the seal 150 can also separate parts of one or more electrical connections between the vaporizer body 110 and the vaporizer cartridge 120. Such arrangements of the seal 150 in the vaporizer device 100 can be helpful in mitigating against potentially disruptive impacts on vaporizer components resulting from interactions with environmental factors (such as water in the vapor or liquid phases, other fluids such as the vaporizable material 102, and/or the like) and/or to reduce the escape of air from the designated airflow path in the vaporizer device 100. Unwanted air, liquid or other fluid passing and/or contacting circuitry of the vaporizer device 100 can cause various unwanted effects, such as altered pressure readings, and/or can result in the buildup of unwanted material, such as moisture, excess vaporizable material 102, etc., in parts of the vaporizer device 100 where they can result in poor pressure signal, degradation of the sensor(s) 113 or other components, and/or a shorter life of the vaporizer device 100. Leaks in the seal 150 can also result in a user inhaling air that has passed over parts of the vaporizer device 100 containing, or constructed of, materials that may not be desirable to be inhaled.

In some implementations, a vaporizer body 110 includes a controller 104, a power source 112 (e.g., battery), one more sensors 113, charging contacts (such as those for charging the power source 112), a seal 150, and a cartridge receptacle 118 configured to receive the vaporizer cartridge 120 for coupling with the vaporizer body 110 through one or more of a variety of attachment structures. In some examples, the vaporizer cartridge 120 includes a reservoir 140 for containing a vaporizable material 102, and a mouthpiece 130 has an aerosol outlet for delivering an inhalable dose to a user. The vaporizer cartridge 120 can include an atomizer 141 having a wicking element and a heating element, or alternatively, one or both of the wicking element and the heating element can be part of the vaporizer body 110. In implementations in which any part of the atomizer 141 (e.g., heating element and/or wicking element) is part of the vaporizer body 110, the vaporizer device 100 can be configured to supply vaporizable material 102 from the reservoir 140 in the vaporizer cartridge 120 to the part(s) of the atomizer 141 included in the vaporizer body 110.

Cartridge-based configurations for the vaporizer device 100 that generate an inhalable dose of a non-liquid vaporizable material 102, via heating of a non-liquid vaporizable material, are also within the scope of the current subject matter. For example, the vaporizer cartridge 120 can include a mass of a plant material that is processed and formed to have direct contact with parts of one or more resistive heating elements, and the vaporizer cartridge 120 can be configured to be coupled mechanically and/or electrically to the vaporizer body 110 that includes the controller 104, the power source 112, and one or more receptacle contacts 125a and 125b configured to connect to one or more corresponding cartridge contacts 124a and 124b and complete a circuit with the one or more resistive heating elements.

In an implementation of the vaporizer device 100 in which the power source 112 is part of the vaporizer body 110, and a heating element is disposed in the vaporizer cartridge 120 and configured to couple with the vaporizer body 110, the vaporizer device 100 can include electrical connection features (e.g., means for completing a circuit) for completing a circuit that includes the controller 104 (e.g., a printed circuit board, a microcontroller, or the like), the power source 112, and the heating element (for example, a heating element within the atomizer 141). These features can include at least two contacts (referred to herein as cartridge contacts 124a and 124b) on a bottom surface of the vaporizer cartridge 120 and at least two contacts (referred to herein as receptacle contacts 125a and 125b) disposed near a base of the cartridge receptacle 118 of the vaporizer device 100 such that the cartridge contacts 124a and 124b and the receptacle contacts 125a and 125b make electrical connections when the vaporizer cartridge 120 is inserted into and coupled with the cartridge receptacle 118. The circuit completed by these electrical connections can allow delivery of electrical current to a heating element and can further be used for additional functions, such as for measuring a resistance of the heating element for use in determining and/or controlling a temperature of the heating element based on a thermal coefficient of resistivity of the heating element.

In some implementations of the current subject matter, the at least two cartridge contacts 124a and 124b and the at least two receptacle contacts 125a and 125b can be configured to electrically connect in either of at least two orientations. In other words, one or more circuits necessary for operation of the vaporizer device 100 can be completed by insertion of the vaporizer cartridge 120 in the cartridge receptacle 118 in a first rotational orientation (around an axis along which the end of the vaporizer cartridge 120 is inserted into the cartridge receptacle 118 of the vaporizer body 110) such that a first cartridge contact 124a is electrically connected to a first receptacle contact 125a and a second cartridge contact 124b is electrically connected to a second receptacle contact 125b. Furthermore, the one or more circuits necessary for operation of the vaporizer device 100 can be completed by insertion of the vaporizer cartridge 120 in the cartridge receptacle 118 in a second rotational orientation such that the first cartridge contact 124a is electrically connected to the second receptacle contact 125b and the second cartridge contact 124b is electrically connected to the first receptacle contact 125a.

In one example of an attachment structure for coupling the vaporizer cartridge 120 to the vaporizer body 110, the vaporizer body 110 includes one or more detents (e.g., dimples, protrusions, etc.) protruding inwardly from an inner surface of the cartridge receptacle 118, additional material (such as metal, plastic, etc.) formed to include a portion protruding into the cartridge receptacle 118, and/or the like. One or more exterior surfaces of the vaporizer cartridge 120 can include corresponding recesses (not shown in FIG. 1A) that can fit and/or otherwise snap over such detents or protruding portions when an end of the vaporizer cartridge 120 is inserted into the cartridge receptacle 118 on the vaporizer body 110. When the vaporizer cartridge 120 and the vaporizer body 110 are coupled (e.g., by insertion of an end of the vaporizer cartridge 120 into the cartridge receptacle 118 of the vaporizer body 110), the detents or protrusions into the cartridge receptacle 118 can fit within and/or otherwise be held within the recesses of the vaporizer cartridge 120, to hold the vaporizer cartridge 120 in place when assembled. Such an assembly can provide enough support to hold the vaporizer cartridge 120 in place to ensure good contact between the at least two cartridge contacts 124a and 124b and the at least two receptacle contacts 125a and 125b, while allowing release of the vaporizer cartridge 120 from the vaporizer body 110 when a user pulls with reasonable force on the vaporizer cartridge 120 to disengage the vaporizer cartridge 120 from the cartridge receptacle 118.

Further to the discussion above about the electrical connections between a vaporizer cartridge 120 and a vaporizer body 110 being reversible such that at least two rotational orientations of the vaporizer cartridge 120 in the cartridge receptacle 118 are possible, in some vaporizer devices 100 the shape of the vaporizer cartridge 120, or at least a shape of the end of the vaporizer cartridge 120 that is configured for insertion into the cartridge receptacle 118 may have rotational symmetry of at least order two. In other words, the vaporizer cartridge 120 or at least the insertable end of the vaporizer cartridge 120 may be symmetric upon a rotation of 180° around an axis along which the vaporizer cartridge 120 is inserted into the cartridge receptacle 118. In such a configuration, the circuitry of the vaporizer device 100 may support identical operation regardless of which symmetrical orientation of the vaporizer cartridge 120 occurs.

In some examples, the vaporizer cartridge 120, or at least an end of the vaporizer cartridge 120 configured for insertion in the cartridge receptacle 118, can have a non-circular cross section transverse to the axis along which the vaporizer cartridge 120 is inserted into the cartridge receptacle 118. For example, the non-circular cross section can be approximately rectangular, approximately elliptical (e.g., have an approximately oval shape), non-rectangular but with two sets of parallel or approximately parallel opposing sides (e.g., having a parallelogram-like shape), or other shapes having rotational symmetry of at least order two. In this context, approximately having a shape indicates that a basic likeness to the described shape is apparent, but that sides of the shape in question need not be completely linear and vertices need not be completely sharp. Rounding of both or either of the edges or the vertices of the cross-sectional shape is contemplated in the description of any non-circular cross section referred to herein.

The at least two cartridge contacts 124a and 124b and the at least two receptacle contacts 125a and 125b can take various forms. For example, one or both sets of contacts can include conductive pins, tabs, posts, receiving holes for pins or posts, or the like. Some types of contacts can include springs or other features to facilitate better physical and electrical contact between the contacts on the vaporizer cartridge 120 and the vaporizer body 110. The electrical contacts can optionally be gold-plated, and/or can include other materials.

FIG. 1B illustrates an implementation of the vaporizer body 110 having a cartridge receptacle 118 into which the vaporizer cartridge 120 can be releasably inserted. FIG. 1B shows a top view of the vaporizer device 100 illustrating the vaporizer cartridge 120 positioned for insertion into the vaporizer body 110. When a user puffs on the vaporizer device 100, air can pass between an outer surface of the vaporizer cartridge 120 and an inner surface of the cartridge receptacle 118 on the vaporizer body 110. Air can then be drawn into the insertable end 122 of the cartridge, through the vaporization chamber that includes or contains the heating element and wick, and out through an outlet of the mouthpiece 130 for delivery of the inhalable aerosol to a user. The reservoir 140 of the vaporizer cartridge 120 can be formed in whole or in part from translucent material such that a level of the vaporizable material 102 is visible within the vaporizer cartridge 120.

Smoking combustible cigarettes leads to higher levels of carbon monoxide (CO) or carboxyhemoglobin (HbCO, the complex formed by an oxygen receptor on a blood cell and a CO molecule) in a user's blood. Thus, CO levels in users' blood or breath can be strong indicators of combustible cigarette use. For example, CO levels of 3% or higher, and/or 9 ppm or higher, can indicate that the user is smoking combustible cigarette(s) and/or has smoked combustible cigarette(s) recently. CO levels of 2.5% or lower, and/or 8 ppm or lower, can indicate that the user has abstained from smoking combustible cigarettes for 24 hours or longer. Some users may have higher than usual CO levels, such that magnitude alone is insufficient to determine whether a user has consumed a combustible cigarette (e.g., false positive). Accordingly, CO levels over time, gross motion of a user's hand, and/or the like can also help to track combustible cigarette use.

Smoking combustible cigarettes can also lead to other health issues, such as increased resting heart rate, increased blood pressure, decreased blood oxygen, increased breathing rate, lack of sleep, other biomarkers, and/or the like. Accordingly, as users transition off of combustible cigarettes, they may also wish to monitor the changes over time to their resting heart rate, blood pressure, other biomarkers, and/or the like.

Accordingly, it may be desirable to monitor one or more user biomarkers, such as a user's CO levels (e.g., in a user's blood, breath, sweat, and/or the like), gross motion of hand, blood oxygen levels, vital signs (e.g., heart rate, breathing rate, and/or the like), activity rate, sleep indicators, and/or the like. While systems and methods described herein refer to monitoring and measuring CO levels of the user, the systems and methods described herein can additionally or alternatively measure one or more other biomarkers of the user, such as the user's gross motion of hand, blood oxygen levels, vital signs (e.g., heart rate, breathing rate, and/or the like), activity rate, sleep indicators, and/or the like.

Active monitoring techniques, such as breathalyzer devices, pulse oximeters, blood sampling, and the like, can be used to monitor CO levels, but require the user to take active steps to measure and monitor the user's CO levels, outside of the ordinary use of vaporizer devices 100. Additionally, actively monitoring CO levels only provides information at discrete points in time, and isolated from any data on the use of the vaporizer device 100. Thus, active methods of measuring and monitoring users' CO levels can discourage the users from tracking their CO levels (e.g., by including additional steps to be taken by the user), and do not provide a complete picture of users' progress to eliminate their use of combustible cigarettes (e.g., by only providing raw CO data).

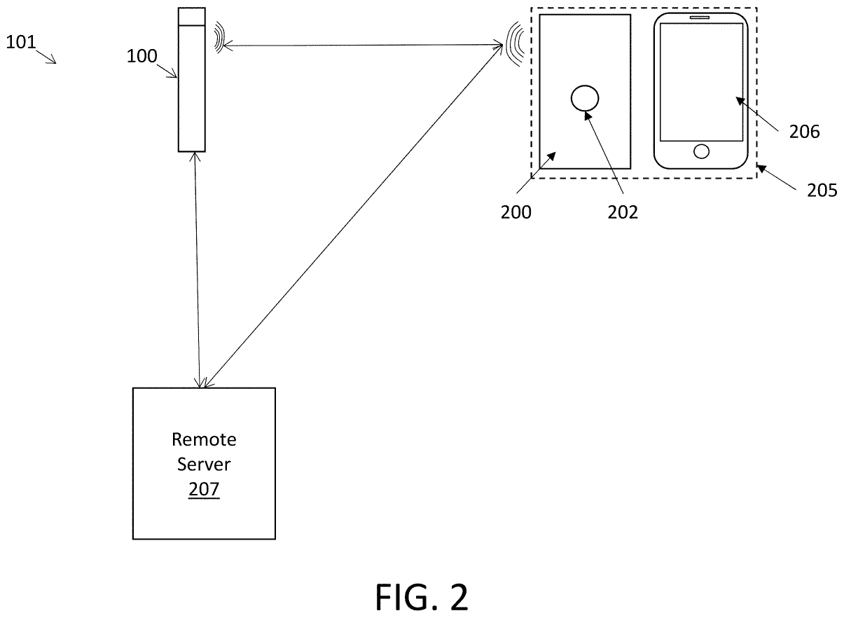

Accordingly, additionally or alternatively to active methods of measuring and monitoring users' CO levels, passive measuring and monitoring systems can be implemented consistent with implementations of the current subject matter. For example, FIG. 2 shows a schematic representation of a vaporizer system 101 including a vaporizer device 100, a user device 205 that communicates (e.g., wirelessly) with the vaporizer device 100, and a remote server 207 that can communicate directly with the vaporizer 100 and/or through the user device 205. The user device 205 can include one or more user devices 205, such as a vaporizer accessory 200, computing device 206 (e.g., a hand-held mobile device such as a smartphone, smartwatch, tablet, and/or the like, a desktop, a laptop, a dedicated remote control device, and/or the like). In some implementations, the one or more user devices 205 can wirelessly communicate with the vaporizer device 100, the remote server 207, and/or with one another. The vaporizer accessory 200, which can be physically separate from, configured for coupling to, or integrated with the vaporizer device 100, can passively monitor CO levels of the user. In some implementations, the vaporizer accessory 200 can include one or more features that require active participation by the user, such as interaction with one or more user interfaces of the vaporizer accessory 200 to set up the vaporizer accessory 200 for use with the vaporizer device 100 and/or computing device 206, start monitoring, stop monitoring, and/or the like.

The vaporizer accessory 200 can include one or more sensors 202. The one or more sensors 202 can include optical sensor(s), touch activated sensor(s), ambient air sensor(s), inhalation sensor(s), exhalation sensor(s), one or more gas sensor(s) (e.g., one or more gas sensors and/or one or more types of gas sensors), such as an integrated microelectromechanical systems (MEMS)-based gas sensor, a combustible gas sensor, a photoionization detector, an infrared sensor, an ultrasonic sensor, an electrochemical gas sensor, a semiconductor sensor, and/or the like, for measuring various biomarkers of the user. The one or more sensors 202, can be positioned on or coupled (e.g., electrically connected, electronically connected, physically connected, wirelessly connected, and/or the like) to a controller (e.g., a printed circuit board assembly or other type of circuit board) on the vaporizer device 100 and/or the vaporizer accessory 200.

Examples of the vaporizer accessory 200 consistent with implementations of the current subject matter described herein can enable the one or more sensors 202 to continuously and/or periodically, actively and/or passively, measure and/or monitor the user's biomarkers (e.g., CO levels), either with or without requiring the user to perform an active step. For example, in some implementations, the user can be prompted to inhale or exhale a certain number of times, while holding the vaporizer device 100 and/or vaporizer accessory 200 in a certain proximity from the user's mouth, lips, and/or other body part. In certain implementations, the user can be prompted to touch or hold the vaporizer device 100 and/or vaporizer accessory 200 at a certain location, for a certain duration of time, in a certain position and/or at a certain angle (e.g., with respect to X, Y, and/or Z axes), and/or the like. For example, the user may be instructed to hold the vaporizer device 100 and/or vaporizer accessory 200 in a certain position and/or at a certain angle until certain vital and/or biomarker signs or signals are measured or collected.

The vaporizer system 101 can use the data acquired (e.g., during the measuring and/or monitoring of the user's CO levels) to inform, assist, or otherwise guide the user, such as users participating in a cessation program. In some implementations, the vaporizer system 101 can notify the user of various statistics based on the monitored biomarkers that may be useful for the user to, for example, track the user's usage of combustible cigarettes, usage of the vaporizer device 100, changes in biomarkers over time, and/or the like. In some implementations, the vaporizer system 101 can proactively notify the user that a relapse (e.g., use of one or more combustible cigarettes) has occurred or may be occurring.

Generally, combustible cigarette and/or vaporizer device 100 usage tracking and data management can be beneficial to the user. In some implementations, the specific data collected by at least the one or more sensors 202 can help determine if changes need to be made to the vaporizer device 100, either manually by the user, or automatically, based on specific real-time, minimum, maximum, or averages of data gathered, or if changes need to be made by the user, for example, in their daily routine. The vaporizer accessory 200 can provide the vaporizer system 101 with the ability to recognize and/or communicate to the user, various metrics and/or patterns, such as when the user is stepping down their cigarette and/or vaporizer device 100 usage, how much the user has used of their daily, weekly, and/or monthly allotment of puffs, nicotine, material vaporized, and/or the like, how often the user has inhaled from a combustible cigarette and/or a vaporizer device 100, how many combustible cigarettes the user has used, and/or the like. Additionally and/or alternatively, other types of usage tracking and management, user interfaces related to usage tracking and management, and/or the like can be implemented, such as those described in more detail in U.S. Provisional Application Nos. 62/793,889, filed on Jan. 17, 2019, and 62/690,271, filed on Jun. 26, 2018, each of which are incorporated by reference herein in their entirety, to the extent permissible. The vaporizer system 101 described herein can also be used to aid with nicotine cessation programs and methods, such as the nicotine cessation programs and methods described in U.S. Provisional Application Nos. 62/793,889, filed on Jan. 17, 2019, and 62/690,271, filed on Jun. 26, 2018, each of which is incorporated by reference herein in their entirety, to the extent permissible.

As illustrated schematically in FIG. 2, any of the vaporizer apparatuses described herein (such as the vaporizer device 100) can remotely communicate with the remote server 207 and/or the user device 205. Thus, the vaporizer device 100 and/or the user device 205 can include a communications hardware 34 that can be implemented through a communication chip (e.g., second communication hardware) in or on the vaporizer device 100. Exemplary wireless chips include, but are not limited to, a Bluetooth chip, such as Parani BCD 210 or Texas Instruments (TI) CC2650 Bluetooth Single-Chip Solution, an NFC-enabled chip (such as Qualcomm's QCA1990), that allows for NFC communication, or enhanced Wi-Fi or Bluetooth communication where NFC is used for link setup. A wireless communication chip can include a Wi-Fi-enabled chip, such as TI's SimpleLink family's CC3000, that can hook the apparatus to Wi-Fi networks. In some implementations, the wireless chip(s) include a subscriber identity module (SIM) card on board of the vaporizer device 100 and/or user device 205, a Nano-SIM card, and/or the like (e.g., allowing 3G/4G/5G cellular network communication). Alternative forms of communication can be used to establish and/or conduct two-way or one-way communications between a vaporizer device 100 and the user device 205. Connection between the vaporizer device 100 and the user device 205 can be automatic (e.g., after an initial set-up), can be initiated by the user through various settings, can be initiated by shaking the vaporizer device 100, and/or the like.

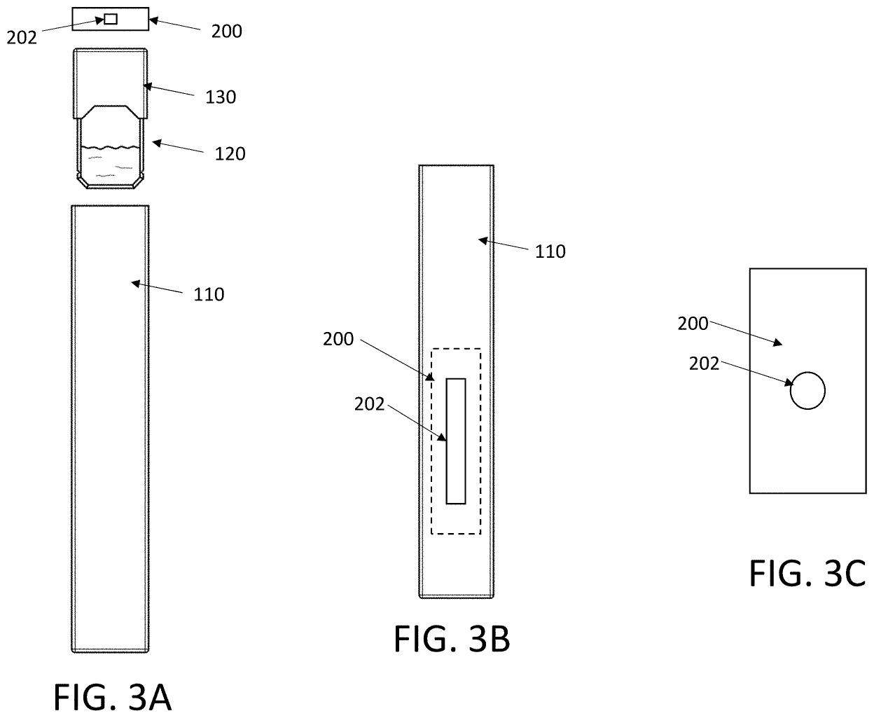

As noted above, the vaporizer accessory 200 can be physically separate from, configured for coupling to, and/or integrated with the vaporizer device 100 to monitor (e.g., passively and/or actively monitor) biomarkers, such as CO levels, of the user. FIGS. 3A-3C illustrate example configurations of the vaporizer accessory 200 consistent with implementations of the current subject matter.

FIG. 3A illustrates an example of the vaporizer accessory 200 that can be detachably connected to the vaporizer device 100. As shown, the vaporizer accessory 200 including one or more integrated sensors 202 can be attached to the vaporizer device 100, such as on the mouthpiece 130. This configuration would allow the one or more sensors 202 to be located on or within the vaporizer device to contact the user's lips when the user takes a puff, and/or to measure CO levels based on the user's breath (e.g., as the user takes the puff).

Thus, the one or more sensors 202 can be positioned such that the one or more sensors 202 can measure and/or monitor the user's CO levels in the ordinary course of using the vaporizer device 100. This allows the sensors 202 to continuously and/or periodically, passively measure and/or monitor the user's CO levels, at least while the user is using the vaporizer device 100. Such configuration can also enable the one or more sensors 202 to continuously and/or periodically, passively measure and/or monitor the user's CO levels without requiring the user to perform an additional active step to measure their CO levels. The vaporizer system 101 can use the data acquired during the measuring and/or monitoring of the user's CO levels to inform and/or assist the user, such as users participating in a cessation program, as described herein.

FIG. 3B illustrates an example of the vaporizer accessory 200 (and/or sensor 202) that is integrated with the vaporizer device 100. As shown, the vaporizer accessory 200 including the one or more integrated sensors 202 can be located along or extending from an outer surface of the vaporizer device 100. For example, the vaporizer accessory 200 can be positioned on the vaporizer device 100 such that the one or more sensors 202 are accessible to the user's hand (and/or fingers) when the user grips the vaporizer device 100, such as when the user is taking a puff

Thus, the one or more sensors 202 can be positioned such that the user's skin contacts the one or more sensors 202 in the ordinary course of using the vaporizer device 100. Such a configuration can allow the sensors 202 to continuously and/or periodically, passively measure and/or monitor the user's biomarker signals (e.g., CO or blood oxygen levels, heart rate, breathing rate, activity rate, and/or the like) at least while the user is using the vaporizer device 100. Such configuration can also enable the one or more sensors 202 to continuously and/or periodically, passively measure and/or monitor the user's biomarkers (e.g., CO levels) without requiring the user to perform an additional active step to measure their biomarkers. The vaporizer system 101 can use the data acquired during the measuring and monitoring of the user's biomarkers to inform and/or assist the user, such as users participating in a cessation program, as described herein.

FIG. 3C illustrates an example of the vaporizer accessory 200, including an integrated sensor 202, that is separate or separable from the vaporizer device 100, consistent with implementations of the current subject matter. For example, the vaporizer accessory 200 can include one or more wearable accessories including one or more integrated sensors 202, such as a band that wraps around the user's arm or leg, a smartwatch, smartwear, a ring, an in-ear or on-ear device (e.g., a headphone, glasses, and/or the like), and/or the like, that can continuously, semi-continuously, and/or periodically be in contact with the user's skin when worn by the user.

Accordingly, the sensors 202 can consistently, continuously and/or periodically, passively measure and/or monitor the user's CO levels. Such configurations can also enable the one or more sensors 202 to either actively or passively measure and/or monitor the user's CO levels with or without requiring the user to perform an additional active step to measure their CO levels. As noted above, the vaporizer accessory 200 can wirelessly communicate with the vaporizer device 100 and/or another user device 205, such as a hand-held mobile device 206. The vaporizer system 101 can use the data acquired during the measuring and monitoring of the user's CO levels to inform and/or assist the user, such as users participating in a cessation program, as described herein.

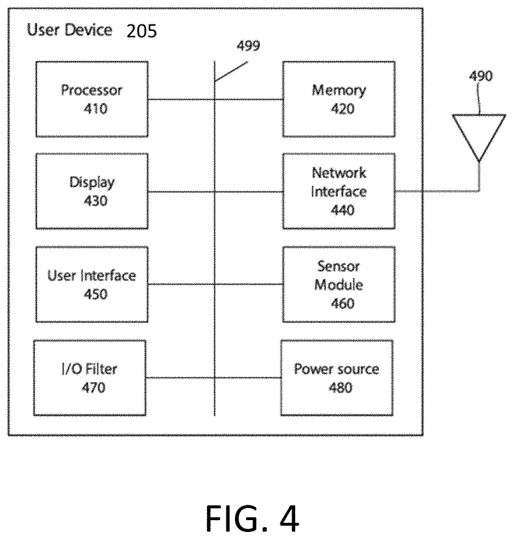

FIG. 4 illustrates an example of the user device(s) 205, which can be used to implement one or more of the described features and/or components, consistent with some example implementations. The user device 205 can perform one or more of the processes described herein. For example, the user device 205 can be used to execute an application providing for user control of a vaporizer device 100 in communication with the user device 205 and/or to provide an interface for the user to engage and interact with functions related to the vaporizer device, in accordance with some example implementations. In some implementations, the user device 205 can be used to execute an application providing for measuring and/or monitoring biomarkers (e.g., CO levels) of the user, to communicate with one or more other user devices 205, the vaporizer device 100, and/or the remote server 207, to provide an interface for the user to engage and interact with functions related to the vaporizer device 100 or usage and/or monitoring management, and/or the like, consistent with implementations described herein.

As illustrated, the user device 205 can include one or more data processors (or controllers) such as processor 410 to execute instructions that can implement operations consistent with those described herein. The user device 205 can include memory 420 to store executable instructions and/or information. The memory 420 can include solid-state memory, solid-state disk drives, magnetic disk drives, or any other information storage device. In some aspects, the memory 420 can provide storage for at least a portion of a database. The user device 205 can include a network interface 440 to a wired network or a wireless network. In order to effectuate wireless communications, the network interface 440, for example, can utilize one or more antennas, such as antenna 490.

The user device 205 can include one or more user interfaces, such as a user interface 450. The user interface 450 can include hardware or software interfaces, such as a keyboard, mouse, or other interface, some of which can include a touchscreen integrated with a display 430. The display 430 can be used to display information, such as information related to the functions of the vaporizer device and/or functions of the vaporizer accessory, provide prompts to a user, receive user input, and/or the like. In various implementations, the user interface 450 can include one or more peripheral devices and/or the user interface 450 can be configured to communicate with these peripheral devices.

In some implementations, the user interface 450 can include one or more of the sensors described herein and/or can include an interface to one or more of the sensors described herein (e.g., the one or more sensors 202). The operation of these sensors can be controlled at least in part by a sensor module 460. The user device 205 can also include an input and output filter 470, which can filter information received from the sensors or other user interfaces, received and/or transmitted by the network interface 440, and/or the like. For example, signals detected through the sensors can be passed through the filter 470 for proper signal conditioning, and the filtered data can then be passed to the sensor module 460 and/or processor 410 for validation and processing (e.g., before transmitting results or an indication via the network interface 440). The user device 205 can be powered through the use of one or more power sources, such as a power source 480. As illustrated, one or more of the components of the user device 205 can communicate and/or receive power through a system bus 499.

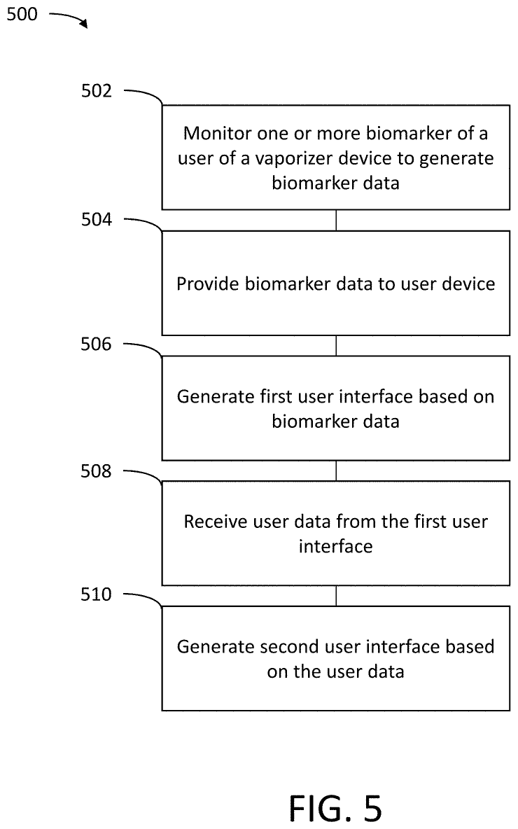

FIG. 5 illustrates an example method 500 for guiding a user, such as a user participating in a nicotine and/or combustible cigarette cessation program. For example, use of the vaporizer accessory 200 to monitor the user's biomarkers (e.g., CO levels) can help to incentivize users to participate or continue participating in a cessation program.

At 502, the one or more sensors 202 of the vaporizer accessory 200 can monitor one or more biomarkers of a user to generate biomarker data. The one or more biomarkers can include a concentration of carbon monoxide in the user's blood, and/or any of the other biomarkers described herein. At 504, the vaporizer accessory 200 can provide (e.g., store and/or transmit), to the user device 205, the biomarker data. In some implementations, the user device 205 is in wireless communication with the vaporizer accessory 200, as described herein, and/or can include a user interface and/or display 430. At 506, the user device 205 can generate and/or display a first user interface to the user, such as via the display 430, based on the biomarker data. In some implementations, the first user interface includes an indication that notifies the user that the one or more biomarkers has exceeded a certain threshold, the concentration of the one or more biomarkers, prompts the user to indicate whether they have smoked a combustible cigarette and how many within a given time period (e.g., within the day, since the last time combustible cigarette was logged, and/or the like), and/or the like.

At 508, the user device 205 can receive user data from the first user interface, such as an indication of whether the user has smoked combustible cigarette(s), when the combustible cigarette(s) were smoked, how many combustible cigarette(s) were smoked within a given time period, and/or the like. For example, the user may indicate that they smoked two combustible cigarettes since the last time they logged combustible cigarettes in the same day. The user device 205 can then process and/or store the user data, which can include transmitting at least a portion of the user data to a remote server 207. In some implementations, the user data obtained over time can be used to train a machine learning or other artificial intelligence algorithm, which can better predict when users have consumed combustible cigarettes based on monitored CO levels and/or other biomarker data. For example, it may be the case that certain users exhibit specific patterns of changes in CO levels over time when they smoke cigarettes, which can be identified and used to better predict combustible cigarette usage.

At 510, based on the user data and/or additional data related to the user (e.g., the biomarker data), the user device 205 can generate and/or display a second user interface to the user, such as via the display 430. For example, the second user interface can display the user's progress in the cessation program, such as by displaying combustible cigarette usage, resting heart rate, blood pressure, blood oxygen, breathing rate, sleep, other biomarkers, and/or the like over time. Ideally, this user interface can provide the user with positive reinforcement that their health is improving based on their reduced combustible cigarette usage and encourage the user to continue to reduce their combustible cigarette usage.

In some implementations, the vaporizer device 100 can provide the user with feedback on one or more of the biomarkers, such as by illuminating one or more LEDs according to a specific pattern. For example, the vaporizer device 100 can illuminate LEDs in a specific pattern to indicate that the user's CO levels indicate that the user smoked a combustible cigarette, a number of combustible cigarettes within a given time, and/or the like. In accordance with such implementations, the vaporizer device can obtain biomarker data from the sensors 202, with or without the use of a user device 205.

In some implementations, the user may be using a vaporizer device 100 as part of their cessation of combustible cigarette usage. If the vaporizer device 100 usage is tracked by the user device 205, then the vaporizer device 100 usage can be included as part of the second user interface.

In some implementations, the indicator includes an incentive to the user for participating in the cessation program (e.g., incentives and/or rewards from sponsors for reducing nicotine intake). Accordingly, the vaporizer system 101 can use the data acquired during the measuring and monitoring of the user's CO levels to inform and/or assist the user, such as users participating in the cessation program. In some implementations, the vaporizer system 101 can notify the user that a relapse (e.g., use of one or more combustible cigarettes) has occurred or may be occurring.

In accordance with one or more implementations, in the following, examples of information that can be provided to the vaporizer system 101 and the results that can be generated or provided to a user by the vaporizer system 101 are disclosed. It is noteworthy that the provided disclosure is by way of example and should not be construed as limiting the disclosed subject matter to the particular details or example implementations. In certain implementations, any vital signs, habits, behaviors or expressions of a user (whether conscious or subconscious) can be monitored to help provide meaningful information to the vaporizer system 101 about the user or the user's surroundings and conditions.

In one example, when a user inhales aerosol (which may or may not contain an active ingredient like nicotine) by puffing on the vaporizer device, aerosol delivery could be modulated based on a measure of particulate content (e.g., total particulate mass (TPM) per puff or TMP per use session). Additionally, notifications or information about device usage and measured user-outputs can be taken into consideration. TPM delivered per puff or per session and the delivery frequency can be modulated by, for example, time of the day, day of the week, user-programmed settings, user-programmed schedule, user's calendar setting, or dynamic response or adaptive schedule based on user data collected.

As provided in further detail below, data collected from the user by the vaporizer system 101 can include biomarkers or data associated with user activity as measured by the vaporizer device, mobile phone, smart watch, fitness tracker, or other wearable or non-wearable device. Examples of how the information provided to the vaporizer system 101 can be used include limiting use (or alerting a user to limit intake or use of the vaporizer device 100) when certain changes to one or more of the following example biological (e.g., vital signs and/or other biomarkers) and/or other metrics are detected:

heartbeat,

heart rate,

perspiration,

pupil dilation,

body temperature,

blood sugar levels,

blinking frequency,

blood or breath CO levels,

blood pressure or vasoconstriction,

blood oxygen levels,

breathing rate,

location (e.g., based on GPS signals),

blood alcohol level (e.g., based on breath analysis),

motion-based activity (e.g., based on gross motion of hand, step-count, or

activity-levels like running, walking, sleeping, and/or sitting).

In example implementations, the vaporizer system 101 can limit nicotine consumption, if it is determined that heart rate of the user is too high or too low. For example, if the heart rate is above a certain threshold, it can be assumed that the user is involved in physical activity (e.g., playing sports) and the user's body is in need of oxygen vs. nicotine. Alternatively, if the detected heart rate is below a certain threshold, it can be assumed that the user has been resting or sitting still for too long. Thus, to protect the user or to encourage physical activity, the level of nicotine delivered (or the use of the vaporizer device 100 itself) can be limited.

In other examples, if a high level of perspiration is detected, delivery of nicotine to the user can be monitored and adjusted. If blood or breath CO levels are within certain ranges, the vaporizer system 101 can increase or decrease nicotine delivery. For example, if CO levels suggest the user has regressed from vaping to smoking (e.g., as a feature included in a nicotine step-down program), the user or his doctor or a third party can be notified. In certain implementations, nicotine-blood concentration can be monitored to help an ideal implementation of a vaporizer device that effectively controls nicotine-blood concentrations.

Depending on implementation, the above measures and data can be simultaneously or periodically gathered by the vaporizer system 101. Usage data can be analyzed to yield a better understanding of how device usage relates to user's health, activity and daily conditions. Collected data by the vaporizer device 100 can be based on an opt-in feature offered to users prior to shipping products or can be implemented during clinical trials with user consent. The collected information can be used to provide means for improving user-experiences and the design of the vaporizer device 100 (e.g., make the device smarter or better). Furthermore, certain collected information can be presented to the users in a meaningful manner to help the users change habits or modify use patterns, thereby resulting in an overall positive change in user behavior.

### Terminology

When a feature or element is herein referred to as being “on” another feature or element, it can be directly on the other feature or element or intervening features and/or elements can also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements can be present. In contrast, when a feature or element is referred to as being “directly connected”, “directly attached” or “directly coupled” to another feature or element, there are no intervening features or elements present.

Although described or shown with respect to one implementation, the features and elements so described or shown can apply to other implementations. It will also be appreciated by those of skill in the art that references to a structure or feature that is disposed “adjacent” another feature can have portions that overlap or underlie the adjacent feature.

Terminology used herein is for the purpose of describing particular embodiments and implementations only and is not intended to be limiting. For example, as used herein, the singular forms “a,” “an,” and “the” are intended to include the plural forms as well, unless the context clearly indicates otherwise.

In the descriptions above and in the claims, phrases such as “at least one of” or “one or more of” can occur followed by a conjunctive list of elements or features. The term “and/or” can also occur in a list of two or more elements or features. Unless otherwise implicitly or explicitly contradicted by the context in which it used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

Spatially relative terms, such as “forward”, “rearward”, “under”, “below”, “lower”, “over”, “upper” and the like, can be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device can be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” can be used herein to describe various features/elements (including steps), these features/elements should not be limited by these terms, unless the context indicates otherwise. These terms can be used to distinguish one feature/element from another feature/element. Thus, a first feature/element discussed below could be termed a second feature/element, and similarly, a second feature/element discussed below could be termed a first feature/element without departing from the teachings provided herein.

As used herein in the specification and claims, including as used in the examples and unless otherwise expressly specified, all numbers can be read as if prefaced by the word “about” or “approximately,” even if the term does not expressly appear. The phrase “about” or “approximately” can be used when describing magnitude and/or position to indicate that the value and/or position described is within a reasonable expected range of values and/or positions. For example, a numeric value can have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative implementations are described above, any of a number of changes can be made to various implementations without departing from the teachings herein. For example, the order in which various described method steps are performed can often be changed in alternative implementations, and in other alternative implementations, one or more method steps can be skipped altogether. Optional features of various device and system implementations can be included in some implementations and not in others. Therefore, the foregoing description is provided primarily for exemplary purposes and should not be interpreted to limit the scope of the claims.

One or more aspects or features of the subject matter described herein can be realized in digital electronic circuitry, integrated circuitry, specially designed application specific integrated circuits (ASICs), field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system can include clients and servers. A client and server are generally remote from each other and typically interact through a communication network. The relationship of client and server arises by virtue of computer programs running on the respective computers and having a client-server relationship to each other.

These computer programs, which can also be referred to programs, software, software applications, applications, components, or code, include machine instructions for a programmable processor, and can be implemented in a high-level procedural language, an object-oriented programming language, a functional programming language, a logical programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example, as would a processor cache or other random access memory associated with one or more physical processor cores.

The examples and illustrations included herein show, by way of illustration and not of limitation, specific implementations in which the subject matter can be practiced. As mentioned, other implementations can be utilized and derived there from, such that structural and logical substitutions and changes can be made without departing from the scope of this disclosure. Such implementations of the inventive subject matter can be referred to herein individually or collectively by the term “invention” merely for convenience and without intending to voluntarily limit the scope of this application to any single invention or inventive concept, if more than one is, in fact, disclosed. Thus, although specific implementations have been illustrated and described herein, any arrangement calculated to achieve the same purpose can be substituted for the specific implementations shown. This disclosure is intended to cover any and all adaptations or variations of various implementations. Combinations of the above implementations, and other implementations not specifically described herein, will be apparent to those of skill in the art upon reviewing the above description. Use of the term “based on,” herein and in the claims is intended to mean, “based at least in part on,” such that an unrecited feature or element is also permissible.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail herein, other modifications or additions are possible. In particular, further features and/or variations can be provided in addition to those set forth herein. For example, the implementations described herein can be directed to various combinations and subcombinations of the disclosed features and/or combinations and subcombinations of several further features disclosed herein. In addition, the logic flows depicted in the accompanying figures and/or described herein do not necessarily require the particular order shown, or sequential order, to achieve desirable results. Other implementations can be within the scope of the following claims.

1. A wireless communication device comprising:
a wireless communication interface; and
a controller configured to:
receive sensor data from a vaporizer device or accessory, the sensor data derived from one or more sensors of the vaporizer device or accessory, the sensor
data based on one or more biomarkers of a user of the vaporizer device or accessory;
determine, based on the sensor data, whether the user smoked a combustible cigarette; and
provide, in response to determining that the user smoked a combustible cigarette, an indication that use of combustible cigarettes has been detected.

2.-4. (canceled)

5. The wireless communication device of claim 1, wherein the one or more biomarkers include a heartbeat, a heart rate, a perspiration level, a pupil dilation, a body temperature, a blood sugar level, a blinking frequency, a blood carbon monoxide level, a breath carbon monoxide level, a blood pressure, a blood oxygen level, a breathing rate, a location, a blood alcohol level, and/or a motion.

6. The wireless communication device of claim 1, wherein the controller is further configured to:
determine, based at least on the sensor data, whether the one or more biomarkers of the user exceed a threshold value; and
in response to determining that the one or more biomarkers of the user exceed the threshold value, provide, to the user, a second indication that the one or more biomarkers exceed the threshold value.

7. The wireless communication device of claim 1, wherein the controller is further configured to:
receive, from the user, one or more inputs indicative of the use of combustible cigarettes; and
detect, based at least on the one or more inputs, the use of combustible cigarettes by the user.

8. The wireless communication device of claim 7, wherein the one or more inputs include a second indication that the user used combustible cigarettes, a quantity of combustible cigarettes used, and/or a time when combustible cigarettes are used.

9. The wireless communication device of claim 8, wherein the second indication is received via a user interface displayed at the accessory associated with the vaporizer device.

10. The wireless communication device of claim 1, wherein the first indication is provided via a user interface displayed at the accessory associated with the vaporizer device.

11. The wireless communication device of claim 1, wherein the use of combustible cigarettes is detected based at least on the one or more biomarkers exceeding a threshold value.

12. The wireless communication device of claim 11, wherein the threshold value comprises a blood carbon monoxide level of 3 percent and/or 9 parts per million.

13. The wireless communication of claim 1, wherein the one or more sensors include an optical sensor, a touch activated sensor, an ambient air sensor, an inhalation sensor, an exhalation sensor, a gas sensor, a photoionization detector, an infrared sensor, an ultrasonic sensor, an electrochemical gas sensor, and/or a semiconductor sensor.

14. A vaporizer device system comprising:
a controller configured to:
receive sensor data from a vaporizer device or an accessory, the sensor data derived from one or more sensors of the vaporizer device or accessory, the sensor
data based on one or more biomarkers of a user of the vaporizer device or accessory;
determine, based on the sensor data, whether the user smoked a combustible cigarette; and
provide, in response to determining that the user smoked a combustible cigarette, an indication that use of combustible cigarettes has been detected.

15. The vaporizer device system of claim 14, wherein the one or more biomarkers include a heartbeat, a heart rate, a perspiration level, a pupil dilation, a body temperature, a blood sugar level, a blinking frequency, a blood carbon monoxide level, a breath carbon monoxide level, a blood pressure, a blood oxygen level, a breathing rate, a location, a blood alcohol level, and/or a motion.

16. The vaporizer device system of claim 14, wherein the controller is further configured to:
determine, based at least on the sensor data, whether the one or more biomarkers of the user exceed a threshold value; and
in response to determining that the one or more biomarkers of the user exceed the threshold value, provide a second indication that the one or more biomarkers exceed the threshold value.

17. The vaporizer device system of claim 14, wherein the controller is further configured to:
receive, from the user, one or more inputs indicative of the use of combustible cigarettes; and
detect, based at least on the one or more inputs, the use of combustible cigarettes by the user.

18. The vaporizer device system of claim 17, wherein the one or more inputs include a second indication that the user used combustible cigarettes, a quantity of combustible cigarettes used, and/or a time when combustible cigarettes are used.

19. The vaporizer device system of claim 14, wherein the indication is provided via a user interface displayed at the accessory associated with the vaporizer device.

20. The vaporizer device system of claim 14, wherein the use of combustible cigarettes is detected based at least on the one or more biomarkers exceeding a threshold value.

21. The vaporizer device system of claim 14, wherein the one or more sensors include an optical sensor, a touch activated sensor, an ambient air sensor, an inhalation sensor, an exhalation sensor, a gas sensor, a photoionization detector, an infrared sensor, an ultrasonic sensor, an electrochemical gas sensor, and/or a semiconductor sensor.

22. The vaporizer device system of claim 14, wherein the vaporizer device comprises a wireless communication device and the controller.

23. The vaporizer device system of claim 14, comprising the vaporizer device and a wireless communication device separate from the vaporizer device, the wireless communication device comprising the controller.
